(19) United States
(12) Patent Application Publication
(10) Pub. No.: US 2019/0298708 A1
(43) Pub. Date: Oct. 3, 2019

(54) METHODS OF TREATING SUBJECTS WITH AN ELEVATED NEUROFILAMENT LIGHT CHAIN LEVEL

(71) Applicant: Coherus Biosciences, Inc., Redwood City, CA (US)

(72) Inventor: Sarita K. Jain, Sunnyvale, CA (US)

(73) Assignee: Coherus Biosciences, Inc., Redwood City, CA (US)

(21) Appl. No.: 16/371,652

(22) Filed: Apr. 1, 2019

Related U.S. Application Data
(60) Provisional application No. 62/651,653, filed on Apr. 2, 2018.

(51) Int. Cl. A61K 31/47; G01N 33/68

(52) U.S. Cl. CPC A61K 31/47; G01N 2800/28; G01N 33/6896

(57) ABSTRACT

Provided herein are methods of treating a subject that include selecting a subject having an elevated level of neurofilament light chain protein in a sample including cerebrospinal fluid, blood, serum, or plasma obtained from the subject, as compared to a reference level of neurofilament light chain protein, and administering a pharmaceutical composition including a therapeutically effective amount of a compound of formula (I), or a pharmaceutically acceptable salt, prodrug, or isomer thereof, to the selected subject.

### CROSS-REFERENCE TO RELATED APPLICATIONS

This application claims priority to U.S. Patent Application Ser. No. 62/651,653, filed on Apr. 2, 2018, the entire contents of which are herein incorporated by reference.

### TECHNICAL FIELD

The present invention relates, at least in part, to methods using CHS-131 for the treatment of subjects having an elevated level of neurofilament light chain protein in a sample comprising cerebrospinal fluid, blood, serum, or plasma that has been obtained from the subject (e.g., as compared to a reference level of neurofilament light chain protein).

### BACKGROUND

Neurological disorders can cause permanent and irreversible damage that can affect a patient's quality of life, such as speech, cognitive skills, motor skills, and metabolism. Treatments that would delay the onset of symptoms of a neurological disorder in a subject are desired for patients that are diagnosed as being in the early or middle stages of a neurological disorder.

### SUMMARY

Provided herein are methods of treating a subject that include: selecting a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and administering a pharmaceutical composition including a therapeutically effective amount of a compound of formula (I),

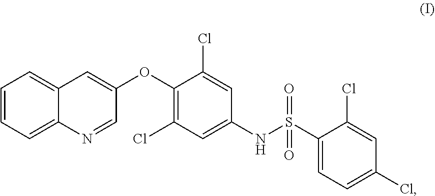 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof, to the selected subject.

Also provided herein are methods of selecting a treatment for a subject that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject. Some embodiments of any of the methods described herein further include administering the selected treatment to the identified subject.

Provided herein are methods of selecting a subject for treatment that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for treatment with a pharmaceutical composition comprising a therapeutically effective amount of a compound of formula (I), or a pharmaceutically acceptable salt, prodrug, or isomer thereof.

Provided herein are methods of selecting a subject for participation in a clinical trial that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that includes administration of a pharmaceutical composition including a therapeutically effective amount of a compound of formula (I), or a pharmaceutically acceptable salt, prodrug, or isomer thereof.

Provided herein are methods of predicting the efficacy of a treatment in a subject that include: determining a level of neurofilament light chain protein level in a sample including cerebrospinal fluid, blood, serum, or plasma obtained from the subject; and determining that a treatment with a pharmaceutical composition including a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein (e.g., any of the exemplary reference levels of neurofilament light chain protein described herein), as compared to a subject not having an elevated level of neurofilament light chain protein in a sample including blood, serum, or plasma as compared to the reference level of neurofilament light chain protein.

In some embodiments of any of the methods described herein, the subject has not been diagnosed with a neurological disorder or neural tissue damage. In some embodiments of any of the methods described herein, the subject does not present with a symptom of a neurological disorder or neural tissue damage (e.g., any of the symptoms of a neurological disorder or neural tissue damage described herein or known in the art). In some embodiments of any of the methods described herein, the subject has been diagnosed as having a neurological disorder (e.g., any of the neurological disorders described herein or known in the art) or neural tissue damage.

Some embodiments of any of the methods described herein further include performing an assay to determine the level of neurofilament light chain protein in the sample obtained from the subject (e.g., any of the exemplary assays for determining a level of neurofilament light chain protein described herein or known in the art). In some embodiments of any of the methods described herein, the assay is a single-molecule array assay.

In some embodiments of any of the methods described herein, the subject has been previously administered a different pharmaceutical composition and the different pharmaceutical composition was determined not to be therapeutically effective.

Also provided herein are methods of determining the efficacy of a treatment in a subject that include: determining a first level of neurofilament light chain protein level in a sample including cerebrospinal fluid, blood, serum, or plasma obtained from the subject at a first time point; determining a second level of neurofilament light chain protein level in a sample including cerebrospinal fluid, blood, serum, or plasma obtained from the subject at a second time point, where the subject received at least one dose of a pharmaceutical composition including a therapeutically effective amount of a compound of formula (I), or a pharmaceutically acceptable salt, prodrug, or isomer thereof, between the first and the second time points; and identifying the pharmaceutical composition as being effective in a subject having a reduced second level of neurofilament light chain protein as compared to the first level of neurofilament light chain protein.

In some embodiments of any of the methods described herein, the subject is a participant in a clinical trial. Some embodiments of any of the methods described herein, the method further includes administering one or more additional doses of the pharmaceutical composition identified as being effective in the subject.

Provided herein are methods of treating a subject that includes: selecting a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subject.

Also provided herein are methods of selecting a treatment for a subject that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject. Some embodiments of any of the methods described herein further include administering the selected treatment to the identified subject.

Also provided herein are methods of selecting a subject for treatment that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject for participation in a clinical trial that include: identifying a subject having an elevated second level of neurofilament light chain protein in a sample providing cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for participation in a clinical trial that includes administration of a pharmaceutical composition including a therapeutically effective amount of a compound of formula (I), or a pharmaceutically acceptable salt, prodrug, or isomer thereof.

In some embodiments of any of the methods described herein, wherein the subject has not been diagnosed with a neurological disorder (e.g., any of the neurological disorders described herein or known in the art) or neural tissue damage. In some embodiments of any of the methods described herein, the subject does not present with a symptom of a neurological disorder or neural tissue damage (e.g., any of the symptoms of a neurological disorder or neural tissue damage described herein or known in the art). In some embodiments of any of the methods described herein, the subject has been diagnosed as having a neurological disorder (e.g., any of the neurological disorders described herein or known in the art) or neural tissue damage.

Some embodiments of any of the methods described herein that includes performing an assay to determine the first level and second levels of neurofilament light chain protein in the sample obtained from the subject at the first time point and the second time point, respectively (e.g., any of the assays for determining a level of neurofilament light chain protein described herein or known in the art). In some embodiments of any of the methods described herein, the assay is a single-molecule array assay.

In some embodiments of any of the methods described herein, the subject has been previously administered a different pharmaceutical composition and the different pharmaceutical composition was determined not to be therapeutically effective. In some embodiments of any of the methods described herein, the subject has not been diagnosed with a neurological disorder (e.g., any of the neurological disorders described herein or known in the art) or neural tissue damage. In some embodiments of any of the methods described herein, the subject does not present with a symptom of a neurological disorder or neural tissue damage (e.g., any of the symptoms of a neurological disorder or neural tissue damage described herein or known in the art).

In one embodiment, the therapeutically effective amount is from about 0.1 to about 15 mg. In another embodiment, the therapeutically effective amount is from about 1 to about 10 mg. In still another embodiment, the therapeutically effective amount is from about 2 to about 6 mg. In yet another embodiment, the therapeutically effective amount is about 3 mg. In another embodiment, the therapeutically effective amount is about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg.

The pharmaceutical compositions used in the methods of the invention may be administered to the subject twice a day, daily, every other day, three times a week, twice a week, weekly, every other week, twice a month, or monthly.

The terms “treat”, “treating” and “treatment” refer to a method of alleviating or abrogating a disease and/or its attendant symptoms. In another embodiment, treating refers to impeding or halting progression of a disease. In yet another embodiment, treating refers to extending the life of a subject with a disease. In some embodiments, treatment can result in a reduction (e.g., an about 1% to about 99% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, or an about 15% to about 20% reduction) in the number, severity, and/or duration of one or more (e.g., two, three, four, five, or six) symptoms and/or metrics (e.g., scores) of a neurological disorder or neural tissue damage (e.g., any of the symptoms and/or metrics of any of the neurological disorders described herein or known in the art).

The term “therapeutically effective amount” refers to that amount of the compound being administered sufficient to treat a disease. In one embodiment, the therapeutically effective amount is sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human. In some embodiments of any of the methods described herein, a subject may be referred to as a patient. In some examples of any of the methods described herein, the subject is 40 years old or older (e.g., 41 years old or older, 42 years old or older, 43 years old or older, 44 years old or older, 45 years old or older, 50 years old or older, 55 years old or older, 60 years old or older, 65 years old or older, 70 years old or older, 75 years old or older, 80 years old or older, 90 years old or older, or 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, or 103 years old).

In some embodiments, the subject does not present with a symptom (e.g., any of the symptoms described herein or known in the art) of a neurological disorder or neural tissue damage (e.g., multiple sclerosis, relapsing-remitting MS (RRMS), clinically isolated syndrome (CIS), primary progressive MS (PPMS), secondary progressive MS (SPMS), or radiologically isolated syndrome (RIS)). In other embodiments, the subject has been diagnosed as having a neurological disorder (e.g., multiple sclerosis, relapsing-remitting MS (RRMS), clinically isolated syndrome (CIS), primary progressive MS (PPMS), secondary progressive MS (SPMS), or radiologically isolated syndrome (RIS)) or neural tissue damage. In other embodiments, the subject has been diagnosed as having neural tissue damage (e.g., severe traumatic brain injury, sports-related mild traumatic brain injury, or post-concussion syndrome). In yet other embodiments, the subject has not been diagnosed as having a neurological disorder or neural tissue damage.

In some embodiments, the subject has a degenerative and traumatic neurological disorder (e.g., dementia, amyotrophic lateral sclerosis, or spinal cord injury).

In yet other embodiments, the subject has been diagnosed or identified as having a neurological disorder that would benefit from treatment with a proliferator-activated receptor gamma (PPARγ) agonist (e.g., CHS-131).

In some embodiments, the subject has previously been administered at least one dose of a CHS-131. In some embodiments, the subject is a participant in a clinical trial.

In other embodiments, the subject has been previously administered a different pharmaceutical composition and the different pharmaceutical composition was determined not to be therapeutically effective.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “biological sample” or “sample” refers to a sample obtained or derived from a subject. By way of example, the sample can include cerebrospinal fluid (CSF), blood, serum, or plasma. In some embodiments, a sample can be, or include, a blood sample. In some embodiments, a sample can be, or include, a serum sample. In some embodiments, a sample can be, or include, a plasma sample.

As used herein, “obtain” or “obtaining” can be any means whereby one comes into possession of the sample by “direct” or “indirect” means. Directly obtaining a sample means performing a process (e.g., performing a physical method such as extraction or phlebotomy) to obtain a sample from the subject. Indirectly obtaining a sample refers to receiving the sample from another party or source (e.g., a third-party laboratory that directly acquired the sample). Thus, obtain is used to mean collection and/or removal of the sample from the subject. Some embodiments of any of them methods described herein can include obtaining a sample or samples from a subject.

The phrase “an elevated” or “an increased level” can be an elevation or an increase of at least 1% (e.g., at least 2%, at least 4%, at least 6%, at least 8%, at least 10%, at least 12%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 115%, at least 120%, at least 140%, at least 150%, at least 200%, at least 250%, at least 300%, at least 350%, at least 400%, or between 1% and 400%, between 1% and 300%, between 1% and 200%, between 1% and 100%, between 1% and 50%, between 1% and 25%, between 1% and 10%, between 10% and 400%, between 10% and 300%, between 10% and 200%, between 10% and 100%, between 10% and 50%, between 50% and 400%, between 50% and 300%, between 50% and 200%, between 50% and 100%, between 50% and 75%, between 75% and 100%, or 1%, 2%, 4%, 5%, 6%, 8%, 10%, 12%, 14%, 16%, 18%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 350%, or 400%), e.g., as compared to a reference level (e.g., any of the exemplary reference levels described herein) or a first level of neurofilament light chain protein.

In some embodiments, an elevated level can be an elevation or an increase of about 1% to about 500%, about 1% to about 450%, about 1% to about 400%, about 1% to about 350%, about 1% to about 300%, about 1% to about 250%, about 1% to about 200%, about 1% to about 150%, about 1% to about 100%, about 1% to about 50%, about 1% to about 25%, about 1% to about 20%, about 1% to about 15%, about 1% to about 10%, about 1% to about 5%, about 2% to about 500%, about 2% to about 450%, about 2% to about 400%, about 2% to about 350%, about 2% to about 300%, about 2% to about 250%, about 2% to about 200%, about 2% to about 150%, about 2% to about 100%, about 2% to about 50%, about 2% to about 25%, about 2% to about 20%, about 2% to about 15%, about 2% to about 10%, about 5% to about 500%, about 5% to about 450%, about 5% to about 400%, about 5% to about 350%, about 5% to about 300%, about 5% to about 250%, about 5% to about 200%, about 5% to about 150%, about 5% to about 100%, about 5% to about 50%, about 5% to about 25%, about 5% to about 20%, about 5% to about 15%, about 5% to about 10%, about 10% to about 500%, about 10% to about 450%, about 10% to about 400%, about 10% to about 350%, about 10% to about 300%, about 10% to about 250%, about 10% to about 200%, about 10% to about 150%, about 10% to about 100%, about 10% to about 50%, about 10% to about 25%, about 10% to about 20%, about 10% to about 15%, about 15% to about 500%, about 15% to about 450%, about 15% to about 400%, about 15% to about 350%, about 15% to about 300%, about 15% to about 250%, about 15% to about 200%, about 15% to about 150%, about 15% to about 100%, about 15% to about 50%, about 15% to about 25%, about 15% to about 20%, about 20% to about 500%, about 20% to about 450%, about 20% to about 400%, about 20% to about 350%, about 20% to about 300%, about 20% to about 250%, about 20% to about 200%, about 20% to about 150%, about 20% to about 100%, about 20% to about 50%, about 20% to about 25%, about 25% to about 500%, about 25% to about 450%, about 25% to about 400%, about 25% to about 350%, about 25% to about 300%, about 25% to about 250%, about 25% to about 200%, about 25% to about 150%, about 25% to about 100%, about 25% to about 50%, about 50% to about 500%, about 50% to about 450%, about 50% to about 400%, about 50% to about 350%, about 50% to about 300%, about 50% to about 250%, about 50% to about 200%, about 50% to about 150%, about 50% to about 100%, about 100% to about 500%, about 100% to about 450%, about 100% to about 400%, about 100% to about 350%, about 100% to about 300%, about 100% to about 250%, about 100% to about 200%, about 100% to about 150%, about 150% to about 500%, about 150% to about 450%, about 150% to about 400%, about 150% to about 350%, about 150% to about 300%, about 150% to about 250%, about 150% to about 200%, about 200% to about 500%, about 200% to about 450%, about 200% to about 400%, about 200% to about 350%, about 200% to about 300%, about 200% to about 250%, about 250% to about 500%, about 250% to about 450%, about 250% to about 400%, about 250% to about 350%, about 250% to about 300%, about 300% to about 500%, about 300% to about 450%, about 300% to about 400%, about 300% to about 350%, about 350% to about 500%, about 350% to about 450%, about 350% to about 400%, about 400% to about 500%, about 400% to about 450%, or about 450% to about 500%, e.g., as compared to a reference level (e.g., any of the exemplary reference levels described herein) or a first level of neurofilament light chain protein.

A “reduced level” can be a 1% to about 99% reduction, a 1% to about 95% reduction, a 1% to about 90% reduction, a 1% to about 85% reduction, a 1% to about 80% reduction, a 1% to about 75% reduction, a 1% to about 70% reduction, a 1% to about 65% reduction, a 1% to about 60% reduction, a 1% to about 55% reduction, a 1% to about 50% reduction, a 1% to about 45% reduction, a 1% to about 40% reduction, a 1% to about 35% reduction, a 1% to about 30% reduction, a 1% to about 25% reduction, a 1% to about 20% reduction, a 1% to about 15% reduction, a 1% to about 10% reduction, a 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 99% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 99% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 99% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 99% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 99% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 99% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 99% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 99% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 99% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 99% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 99% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 99% reduction, an about 70% to about 95% reduction, an about 70% to about 90% reduction, an about 70% to about 85% reduction, an about 70% to about 80% reduction, an about 70% to about 75% reduction, an about 75% to about 99% reduction, an about 75% to about 95% reduction, an about 75% to about 90% reduction, an about 75% to about 85% reduction, an about 75% to about 80% reduction, an about 80% to about 99% reduction, an about 80% to about 95% reduction, an about 80% to about 90% reduction, an about 80% to about 85% reduction, an about 85% to about 99% reduction, an about 85% to about 95% reduction, an about 85% to about 90% reduction, an about 90% to about 99% reduction, an about 90% to about 95% reduction, or an about 95% to about 99% reduction, e.g., in a second level of neurofilament light chain protein as compared to a first level of neurofilament light chain protein.

As used herein a “first time point” can, e.g., refer to an initial time point wherein the subject has not yet received a dose of a pharmaceutical composition (e.g., any of the pharmaceutical compositions described herein). In some examples, a first time point can be, e.g., a time point when a subject has been diagnosed with a neurological disorder or neural tissue damage prior to receiving any treatment (e.g., any of the exemplary treatments described herein). In other examples, a first time point can be a time point when a subject has developed at least one (e.g., 1, 2, 3, 4, 5, 6, 7, 8, 9 or 10) symptom(s) associated with a neurological disorder or neural tissue damage (e.g., any of the exemplary symptoms of a neurological disorder or neural tissue damage described herein or known in the art) and has not received any treatment. In some embodiments, a first time point can represent a time point after which a subject has previously received a different pharmaceutical treatment and the different pharmaceutical treatment was deemed not be successful.

As used herein a “second time point” can, e.g., refer to a second time point after the first time point. In some examples, a subject can receive or has received at least one (e.g., 1, 2, 3, 4, 5, 6, 7, 8, 9 or 10) dose of a pharmaceutical composition (e.g., any of the pharmaceutical compositions) between the first and the second time points. In some embodiments, the time difference between a first and second time point can be, e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 7 months, 8 months 9 months, 10 months, 11 months, or 1 year.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF FIGURES

FIG. 1 shows the relationship between levels of NFL and new CE lesions in subjects administered CHS-131.

FIG. 2 shows the relationship between levels of NFL and new or enlarging T2 lesions in subjects administered CHS-131.

FIG. 3 shows the relationship between levels of NFL and EDSS in subjects administered CHS-131.

### DETAILED DESCRIPTION

Provided are methods of selecting a treatment for a subject that: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject.

Provided herein are methods of selecting a subject for treatment that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for treatment with a pharmaceutical composition including a therapeutically effective amount of a compound of formula (I), or a pharmaceutically acceptable salt, prodrug, or isomer thereof.

Provided herein are methods of selecting a subject for participation in a clinical trial that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that includes administration of a pharmaceutical composition comprising a therapeutically effective amount of a compound of formula (I), or a pharmaceutically acceptable salt, prodrug, or isomer thereof.

Provided herein are methods of predicting the efficacy of a treatment in a subject that include: determining a level of neurofilament light chain protein level in a sample including cerebrospinal fluid, blood, serum, or plasma obtained from the subject; and determining that a treatment with a pharmaceutical composition including a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein (e.g., any of the exemplary reference levels of neurofilament light chain protein described herein), as compared to a subject not having an elevated level of neurofilament light chain protein in a sample including blood, serum, or plasma as compared to the reference level of neurofilament light chain protein.

Provided herein are methods of determining the efficacy of a treatment in a subject that include: determining a first level of neurofilament light chain protein level in a sample including cerebrospinal fluid, blood, serum, or plasma obtained from the subject at a first time point; determining a second level of neurofilament light chain protein level in a sample including cerebrospinal fluid, blood, serum, or plasma obtained from the subject at a second time point, where the subject received at least one (e.g., at least two, at least three, at least four, at least five, at least six, at least seven, at least eight, at least nine, or at least ten) dose of a pharmaceutical composition including a therapeutically effective amount of a compound of formula (I), or a pharmaceutically acceptable salt, prodrug, or isomer thereof, between the first and the second time points; and identifying the pharmaceutical composition as being effective in a subject having a reduced second level of neurofilament light chain protein as compared to the first level of neurofilament light chain protein.

Provided herein are methods of treating a subject that includes: selecting a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subject.

Provided herein are methods of selecting a treatment for a subject that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject.

Also provided herein are methods of selecting a subject for treatment that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject for participation in a clinical trial that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for participation in a clinical trial that includes administration of a pharmaceutical composition including a therapeutically effective amount of a compound of formula (I), or a pharmaceutically acceptable salt, prodrug, or isomer thereof.

### Neurofilament Light Chain Protein and Methods of Detecting Neurofilament Light Chain Protein

Neurofilaments are type IV intermediate filaments and heteropolymers. Neurofilaments are expressed in the brain and are unique to neuronal cells. Neurofilaments in the central nervous system include neurofilament heavy chain (NFH), neurofilament medium chain (NFM), neurofilament light chain (NFL), and α-internexin. Neurofilaments in the peripheral nervous system comprise NFH, NFM, NFL, and peripherin. Neurofilaments are vital for the maintenance of axon caliber, axon radial growth, and the intracellular transport of electrical impulses along axons (Eyer and Peterson, Neuron 12: 389-405, 1994; Ohara et al., J. Cell Biol. 121: 387-395, 1993; Zhu et al., Exp. Neurol. 148: 299-316, 1997).

Mutations in the NFL gene have been shown to cause Charcot-Marie Tooth disease, demyelinating type 1F (CMT1F), and Charcot-Marie Tooth disease, axonal type 2E (CMT2). Mutations in the NFL gene affect the assembly of neurofilaments in neurons (Sasaki et al., Hum. Mol. Genet. 15: 943-952, 2006; and Yates et al., Eur. J. Cell Biol. 88: 193-202, 2009).

Together with the medium and heavy subunits, neurofilament light chain represents one of the scaffolding proteins of the neuronal cytoskeleton (Teunissen et al., Mult. Scler. 18(5):552-556, 2012) and is released in the extracellular space following axonal damage.

The cDNA sequence and protein sequence of human neurofilament light chain is provided in SEQ ID NO: 1 and SEQ ID NO: 2, respectively. The cDNA sequence and protein sequence of mouse neurofilament light chain is provided in SEQ ID NO: 3 and SEQ ID NO: 4, respectively.

Human neurofilament light chain cDNA sequence
(SEQ ID NO: 1)
ATGAGTTCCTTCAGCT ACGAGCCGTA CTACTCGACC TCCTACAAGC
GGCGCTACGT GGAGACGCCCCGGGTGCACA TCTCCAGCGT
GCGCAGCGGC TACAGCACCG CACGCTCAGC
TTACTCCAGCTACTCGGCGC CGGTGTCTTC CTCGCTGTCC
GTGCGCCGCA GCTACTCCTC CAGCTCTGGATCGTTGATGC
CCAGTCTGGA GAACCTCGAC CTGAGCCAGG TAGCCGCCAT
CAGCAACGACCTCAAGTCCA TCCGCACGCA GGAGAAGGCG
CAGCTCCAGG ACCTCAATGA CCGCTTCGCC AGCTTCATCG
AGCGCGTGCA CGAGCTGGAG CAGCAGAACA AGGTCCTGGA
AGCCGAGCTGCTGGTGCTGC GCCAGAAGCA CTCCGAGCCA
TCCCGCTTCC GGGCGCTGTA CGAGCAGGAGATCCGCGACC
TGCGCCTGGC GGCGGAAGAT GCCACCAACG AGAAGCAGGC
GCTCCAGGGCGAGCGCGAAG GGCTGGAGGA GACCCTGCGC
AACCTGCAGG CGCGCTATGA AGAGGAGGTGCTGAGCCGCG
AGGACGCCGA GGGCCGGCTG ATGGAAGCGC GCAAAGGCGC
CGACGAGGCG GCGCTCGCTC GCGCCGAGCT CGAGAAGCGC
ATCGACAGCT TGATGGACGA AATCTCTTTTCTGAAGAAAG
TGCACGAAGA GGAGATCGCC GAACTGCAGG CGCAGATCCA
GTACGCGCAGATCTCCGTGG AGATGGACGT GACCAAGCCC
GACCTTTCCG CCGCGCTCAA GGACATCCGCGCGCAGTACG
AGAAGCTGGC CGCCAAGAAC ATGCAGAACG CTGAGGAATG
GTTCAAGAGCCGCTTCACCG TGCTGACCGA GAGCGCCGCC
AAGAACACCG ACGCCGTGCG CGCCGCCAAG GACGAGGTGT
CCGAGAGCCG TCGTCTGCTC AAGGCCAAGA CCCTGGAAAT
CGAAGCATGCCGGGGCATGA ATGAAGCGCT GGAGAAGCAG
CTGCAGGAGC TGGAGGACAA GCAGAACGCCGACATCAGCG
CTATGCAGGA CACGATCAAC AAATTAGAAA ATGAATTGAG
GACCACAAAGAGTGAAATGG CACGATACCT AAAAGAATAC
CAAGACCTCC TCAACGTGAA GATGGCTTTGGATATTGAGA
TTGCAGCTTA CAGGAAACTC TTGGAAGGCG AGGAGACCCG
ACTCAGTTTC ACCAGCGTGG GAAGCATAAC CAGTGGCTAC
TCCCAGAGCT CCCAGGTCTT TGGCCGATCTGCCTACGGCG
GTTTACAGAC CAGCTCCTAT CTGATGTCCA CCCGCTCCTT
CCCGTCCTACTACACCAGCC ATGTCCAAGA GGAGCAGATC
GAAGTGGAGG AAACCATTGA GGCTGCCAAGGCTGAGGAAG
CCAAGGATGA GCCCCCCTCT GAAGGAGAAG CCGAGGAGGA
GGAGAAGGACAAGGAAGAGG CCGAGGAAGA GGAGGCAGCT
GAAGAGGAAG AAGCTGCCAA GGAAGAGTCT GAAGAAGCAA
AAGAAGAAGA AGAAGGAGGT GAAGGTGAAG AAGGAGAGGA
AACCAAAGAAGCTGAAGAGG AGGAGAAGAA AGTTGAAGGT
GCTGGGGAGG AACAAGCAGC TAAGAAGAAAGATTGA
Human neurofilament light chain amino acid
sequence
(SEQ ID NO: 2)
MSSFSYEPYYSTSYKRRYVETPRVHISSVRSGYSTARSAYSSYSAPVSS
SLSVRRSYSSSSGSLMPSLENLDLSQVAAISNDLKSIRTQEKAQLQDLN
DRFASFIERVHELEQQNKVLEAELLVLRQKHSEPSRFRALYEQEIRDLR
LAAEDATNEKQALQGEREGLEETLRNLQARYEEEVLSREDAEGRLMEAR
KGADEAALARAELEKRIDSLMDEISFLKKVHEEEIAELQAQIQYAQISV
EMDVTKPDLSAALKDIRAQYEKLAAKNMQNAEEWEKSRFTVLTESAAKN
TDAVRAAKDEVSESRRLLKAKTLEIEACRGMNEALEKQLQELEDKQNAD
ISAMQDTINKLENELRTTKSEMARYLKEYQDLLNVKMALDIEIAAYRKL
LEGEETRLSETSVGSITSGYSQSSQVFGRSAYGGLQTSSYLMSTRSEPS
YYTSHVQEEQIEVEETIEAAKAEEAKDEPPSEGEAEEEEKDKEEAEEEE
AAEEEEAAKEESEEAKEEEEGGEGEEGEETKEAEEEEKKVEGAGEEQAA
KKKD
Mouse neurofilament light chain cDNA sequence
(SEQ ID NO: 3)
ATGAGTTCGTTCGGCTACGATCCGTACTTTTCGACCTCCTACAAGCGGC
GCTATGTGGAGACGCCCCGGGTGCACATCTCCAGCGTGCGCAGCGGCTA
CAGCACGGCGCGCTCCGCGTACTCCAGCTACTCCGCGCCGGTCTCCTCC
TCGCTGTCCGTGCGCCGCAGCTACTCGTCCAGCTCTGGCTCTTTGATGC
CCAGCCTGGAGAATCTCGATCTGAGCCAGGTAGCCGCCATCAGCAACGA
CCTCAAGTCTATCCGCACACAAGAGAAGGCACAGCTGCAGGACCTCAAC
GATCGCTTCGCCAGCTTCATCGAGCGCGTGCACGAGCTGGAGCAGCAGA
ACAAGGTCCTGGAAGCCGAGCTGTTGGTGCTGCGCCAGAAACACTCTGA
GCCTTCCCGCTTCCGCGCCCTGTACGAGCAGGAGATCCGCGATCTGCGG
CTGGCAGCGGAAGACGCCACTAACGAGAAGCAGGCGCTGCAGGGCGAGC
GCGAGGGGCTGGAGGAGACTCTGCGCAACCTGCAGGCTCGCTATGAGGA
AGAAGTGCTGAGCCGCGAGGACGCCGAGGGCCGGCTGATGGAAGCGCGC
AAAGGTGCGGATGAGGCCGCGCTCGCCCGCGCCGAGCTGGAGAAGCGCA
TCGACAGCCTGATGGACGAGATAGCTTTCCTGAAGAAGGTGCACGAGGA
AGAGATCGCCGAGCTGCAGGCTCAGATCCAGTATGCTCAGATCTCCGTG
GAGATGGACGTGTCCTCCAAGCCCGACCTCTCCGCCGCTCTCAAGGACA
TCCGCGCTCAGTACGAGAAGCTGGCCGCCAAGAACATGCAGAACGCCGA
AGAGTGGTTCAAGAGCCGCTTCACCGTGCTAACCGAGAGCGCCGCCAAG
AACACCGACGCTGTGCGCGCTGCCAAGGACGAGGTGTCGGAAAGCCGCC
GCCTGCTCAAGGCTAAGACCCTGGAGATCGAAGCCTGCCGGGGTATGAA
CGAAGCTCTGGAGAAGCAGCTGCAGGAGCTAGAGGACAAGCAGAATGCA
GACATTAGCGCCATGCAGGACACAATCAACAAACTGGAGAATGAGCTGA
GAAGCACGAAGAGCGAGATGGCCAGGTACCTGAAGGAGTACCAGGACCT
CCTCAATGTCAAGATGGCCTTGGACATCGAGATTGCAGCTTACAGAAAA
CTCTTGGAAGGCGAAGAGACCAGGCTCAGTTTCACCAGCGTGGGTAGCA
TAACCAGCGGCTACTCTCAGAGCTCGCAGGTCTTCGGCCGTTCTGCTTA
CAGTGGCTTGCAGAGCAGCTCCTACTTGATGTCTGCTCGCTCTTTCCCA
GCCTACTATACCAGCCACGTCCAGGAAGAGCAGACAGAGGTCGAGGAGA
CCATTGAGGCTACGAAAGCTGAGGAGGCCAAGGATGAGCCCCCCTCTGA
AGGAGAAGCAGAAGAGGAGGAGAAGGAGAAAGAGGAGGGAGAGGAAGAG
GAAGGCGCTGAGGAGGAAGAAGCTGCCAAGGATGAGTCTGAAGACACAA
AAGAAGAAGAAGAAGGTGGTGAGGGTGAGGAGGAAGACACCAAAGAATC
TGAAGAGGAAGAGAAGAAAGAGGAGAGTGCTGGAGAGGAGCAGGTGGCT
AAGAAGAAAGATTGA
Mouse neurofilament light chain amino acid
sequence
(SEQ ID NO: 4)
MSSFGYDPYFSTSYKRRYVETPRVHISSVRSGYSTARSAYSSYSAPVSSSLSV
RRSYSSSSGSLMPSLENLDLSQVAAISNDLKSIRTQEKAQLQDLNDRFASFIE
RVHELEQQNKVLEAELLVLRQKHSEPSRFRALYEQEIRDLRLAAEDATN
EKQALQGEREGLEETLRNLQARYEEEVLSREDAEGRLMEARKGADEAAL
ARAELEKRIDSLMDEIAFLKKVHEEEIAELQAQIQYAQISVEMDVSSKP
DLSAALKDIRAQYEKLAAKNMQNAEEWFKSRFTVLTESAAKNTDAVRAA
KDEVSESRRLLKAKTLEIEACRGMNEALEKQLQELEDKQNADISAMQDT
INKLENELRSTKSEMARYLKEYQDLLNVKMALDIEIAAYRKLLEGEETR
LSETSVGSITSGYSQSSQVFGRSAYSGLQSSSYLMSARSFPAYYTSHVQ
EEQTEVEETIEATKAEEAKDEPPSEGEAEEEEKEKEEGEEEEGAEEEEA
AKDESEDTKEEEEGGEGEEEDTKESEEEEKKEESAGEEQVAKKKD

Some embodiments of any of the methods described herein can include a step of performing an assay to determine a level or levels (e.g., first and second level) of neurofilament light chain protein in a sample or samples (e.g., samples obtained from the subject at a first and a second time point). Non-limiting assays that may be used to detect a level or levels of neurofilament light chain are described herein. Additional assays that may be used to detetct a level or levels of neurofilament light chain are known in the art.

A commercially-available enzyme-linked immunosorbent assay (UmanDiagnostics) can be used to measure a level or levels of neurofilament light chain protein in a sample or samples including cerebrospinal fluid, blood, serum, or plasma from a subject. An electrochemiluminescence (ECL)-based assay can also be used to detect a level or levels of neurofilament light chain. See, e.g., Lycke et al., J. Neurol. Neurosurg. Psychiatry 64(3):402-404, 1998; Teunissen et al., Neurology 72(15):1322-1329, 2009; Disanto et al., J. Neurol. Neurosurg. Psychiatry 87(2):126-129, 2015; and Kuhle et al., Mult. Scler. 22(12): 1550-1559, 2016). Another assay that can be performed to detect a level or levels of neurofilament light chain protein is a single-molecule array (Simoa) assay, which is described in detail in Kuhle et al., Clin. Chem. Lab Med. 54(10): 1655-166, 2016; and Gisslen et al., EBioMedicine 3:135-140, 2016). A Simoa assay for neurofilament light chain is commercially available from Quanterix (NF-LIGHT®). The NF-LIGHT® Quanterix Simoa assay has been used to detect levels of neurofilament light chain protein in samples including cerebrospinal fluid, blood, serum, or plasma from human subjects. The antibodies used in the NF-LIGHT® Quanteriz Simoa assay (obtained from Uman Diagnostics, Umea, Sweden) show cross-reactivity with human, mouse, bovine, and macaque neurofilament light chain proteins. The NF-LIGHT® Quanterix Simoa assay is a digital immunoassay.

Additional non-limiting assays that can be used to detect a level of neurofilament light chain protein in a sample include: enzyme-linked immunosorbent assay (ELISA), sensitive sandwich ELISA assay, electrochemiluminescence (ECL)-based assay, mass spectrometry (MS), western blotting, fluorescence-activated cell sorting (FACS), immunohistochemistry.

Other assays that can be used to detect a level or levels of neurofilament light chain in a sample obtained from a subject (e.g., any of the exemplary samples described herein) include immunoassays (e.g., enzyme-linked immunosorbent assays, sandwich enzyme-linked immunosorbent assays, and immunoprecipitation) and proteomic techniques.

### Reference Levels

In some embodiments of any of the methods described herein, the reference level can be a level of neurofilament light chain protein detected in a similar sample obtained from a subject (e.g., a subject who is between 18 to 70 years of age), that has not been diagnosed or identified as having a neurological disorder (e.g. MS) or neural tissue damage, and does not have a family history of a neurological disorder (e.g., MS) or neural tissue damage. In some embodiments, a reference level can be threshold level of neurofilament light chain protein.

A reference level of neurofilament light chain protein (e.g., for samples including serum) is about 10 pg/mL to about 35 pg/mL (e.g., about 10 pg/mL to about 30 pg/mL, about 10 pg/mL to about 25 pg/mL, about 10 pg/mL to about 20 pg/mL, 10 pg/mL to about 15 pg/mL, 15 pg/mL to about 35 pg/mL about 15 pg/mL to about 30 pg/mL, about 15 pg/mL to about 25 pg/mL, about 15 pg/mL to about 20 pg/mL, about 20 pg/mL to about 35 pg/mL, about 20 pg/mL to about 30 pg/mL, about 20 pg/mL to about 25 pg/mL, about 25 pg/mL to about 35 pg/mL, or about 25 pg/mL or about 30 pg/mL).

In some embodiments, a reference level of neurofilament light chain protein can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the levels of neurofilament light chain protein detected in similar samples in a population of healthy subjects (e.g., subjects that are not diagnosed or identified as having a disease (e.g., any of the neurological disorders described herein or neural tissue damage), do not present with a symptom of a disorder or disease (e.g., a neurological disease or disorder), and are not considered to have an elevated risk of developing a neurological disease or disorder).

In some embodiments, a reference level can be the level of neurofilament light chain detected in a similar sample obtained from the subject at an earlier time point.

### CHS-131

CHS-131 is an exemplary compound of formula (I).

In one aspect, CHS-131 reduces neural inflammation in a subject having been diagnosed as having a neurological disorder (e.g., multiple sclerosis (MS), relapsing-remitting MS (RRMS), or Alzheimer's disease) or neural tissue damage, or a subject that does not present with a symptom of a neurological disorder (e.g., multiple sclerosis (MS), relapsing-remitting MS (RRMS), or Alzheimer's disease) or neural tissue damage. In some embodiments, a subject can be identified or diagnosed as having an early stage of a neurological disorder or an early stage or mild neural tissue damage.

In some embodiments, administration of CHS-131 reduces atrophy or degeneration of the brain in the subject (e.g., any of the subjects described herein). In some embodiments, administration of CHS-131 reduces atrophy or degradation of the substantia nigra, globus palladus, subthalamic nucleus and/or cerebellum in the subject (e.g., any of the subjects described herein). In some embodiments, administration of CHS-131 reduces cortical atrophy in the brain of the subject (e.g., any of the subjects described herein).

In one aspect, administration of CHS-131 decreases the risk of developing a comorbidity (e.g., cardiovascular disease, type 2 diabetes mellitus) in the subject (e.g., any of the subjects described herein).

In another embodiment, a period of time during which the therapeutic effects of INT131 (CHS-131) are observed in a subject can be, e.g., 1 week, 2 weeks, 3 weeks, 4 weeks, 1 month, 2 months, 3 months, 4 months, 5 months, 6 months, 7 months, 8 months, 9 months, 10 months, 11 months, 1 year, 1.25 years, 1.5 years, 1.75 years, 2 years, 2.25 years, 2.5 years, 2.75 years, 3 years, 3.5 years, 4 years, 4.5 years, and 5 years.

In one embodiment, CHS-131 can be administered in the form of a besylate salt.

In another embodiment, the therapeutically effective amount is from about 0.1 to about 10 milligrams. In another embodiment, the therapeutically effective amount is from about 1 to about 4 milligrams. In still another embodiment, the therapeutically effective amount is from about 2 to about 3 milligrams. In yet another embodiment, the therapeutically effective amount is about 3 mg, about 4 mg, about 5 mg, about 6 mg, about 7 mg, about 8 mg, about 9 mg, or about 10 mg.

In another embodiment, a composition comprising a therapeutically effective amount of CHS-131 is administered to the subject at an interval that includes, but is not limited to, twice a day, daily, every other day, three times a week, twice a week, weekly, every other week, twice a month, monthly, and every other month.

In another embodiment, a composition comprising a therapeutically effective amount of CHS-131 is administered orally to a subject. In yet another embodiment, the composition is substantially the same as those disclosed in US Patent Application Publication 2013-0243865, the disclosure of which is expressly incorporated herein by reference.

In one embodiment, CHS-131 is as effective, or more effective, treating a neurological disorder or neural tissue damage than other therapies. These therapies include therapies approved for treating a neurological disorder or neural tissue damage (e.g., any of the neurological disorders described herein) and those in development for treating a neurological disorder or neural tissue damage (e.g., any of the neurological disorder described herein). These therapies include, but are not limited to, medications to treat movement disorders, medications to treat psychiatric disorders, psychotherapy, speech therapy, physical therapy, and occupational therapy.

Medications to treat movement disorders include, but are not limited to, tetrabenazine, antipsychotic drugs, such as haloperidol, chlorpromazine, risperidone, and quetiapine, and other medications such as amantadine, levetiracetam, and, clonazepam.

Medications to treat psychiatric disorders include, but are not limited to, antidepressants such as citalopram, fluoxetine, and sertraline, antipsychotic drugs such as quetiapine, risperidone, and olanzapine, and mood-stabilizing drugs, including anticonvulsants, such as valproate, carbamazepine, and lamotrigine.

Psychotherapy includes, but is not limited to, talk therapy to help a subject manage behavioral problems, depression, and suicidal thoughts.

Speech therapy includes, but is not limited to, improving a subjects ability to speak clearly, and improve function and control of muscles used for eating and swallowing.

Physical therapy includes, but is not limited to, enhancing strength, flexibility, balance and coordination, reducing the risk of falls, and improve posture to lessen the severity of movement problems.

Occupational therapy includes, but is not limited to, use of assistive devices that improve functional abilities such as handrails, and eating and drinking utensils for subjects with diminished motor skills.

In another embodiment, CHS-131 is administered to a subject in need thereof in combination with one or more therapies listed herein.

In some embodiments of any of the methods described herein, a pharmaceutical composition including a therapeutically effective amount of formula (I), or a pharmaceutically acceptable salt, prodrug, or isomer thereof, can be co-administered to the subject with one or more additional treatments (e.g., any of the other exemplary treatments or therapies described herein)

### Methods of Treating

Provided herein are methods of treating a subject that include: selecting a subject (e.g., any of the exemplary subjects described herein) having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein or known in the art); and administering a pharmaceutical composition including a therapeutically effective amount of a compound of formula (I), or a pharmaceutically acceptable salt, prodrug, or isomer thereof, to the selected subject (e.g., e.g., CHS-131) using any of the doses or frequencies of administration described herein).

Also provided are methods of treating a subject (e.g., any of the subjects described herein) that include: selecting a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subject (e.g., CHS-131, e.g., using any of the doses or frequencies of administration described herein).

In some embodiments of any of the methods of treatment described herein, the method can result in a decreased risk of developing comorbidity in the subject (e.g., as compared to the risk of developing comorbidity in a similar subject having a similar neurological disorder, but administered a different treatment). Some embodiments of any of the methods described herein can further include administering to the subject an agent that alleviates a negative side effect of a compound of formula (I), or a pharmaceutically acceptable salt, prodrug, or isomer thereof, in the subject (e.g., weight loss or mood swings).

In some embodiments of any of the methods of treatment described herein, the method can result in increasing the life span of the subject (e.g., as compared to a similar subject having a similar neurological disorder, but receiving a different treatment). In some embodiments of any of the methods of treatment described herein, the method results in an improvement in the motor function of the subject (e.g., as compared to the motor function of the subject prior to treatment).

Some embodiments of any of the methods of treatment described herein further can include administering to the subject an agent for treating depression, obsessive-compulsive behavior, and/or apathy. Some embodiments of any of the methods described herein can further include administering to the subject an agent that alleviates eye irritation and/or eye closure symptoms. Some embodiments of any of the methods described herein can further include administering to the subject a treatment for reducing weight loss or a treatment for reducing the risk of developing aspiration pneumonia.

### Methods of Selecting a Treatment for a Subject

Provided herein are methods of selecting a treatment for a subject (e.g., any of the subjects described herein) that include: identifying a subject having an elevated level of neurofilament light chain protein in a sample comprising cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject.

Also provided are methods of selecting a treatment for a subject (e.g., any of the subjects described herein) that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject. In any of the methods that include detecting a level of neurofilament light chain protein in a first and a second sample, one skilled in the art will appreciate that, e.g., the samples should be similar (e.g., both samples are serum samples, both samples are blood samples, both samples are plasma samples, or both samples are cerebrospinal fluid samples).

Some embodiments of these methods further include administering one or more doses (e.g., at least two, at least five, or at least ten doses) of the selected pharmaceutical composition to the identified subject. Some embodiments of these methods further include recording the selected pharmaceutical composition in the identified subject's clinical records.

Also provided herein are methods of selecting a subject for treatment that include: identifying a subject having an elevated level of neurofilament light chain protein in a sample comprising cerebrospinal fluid, blood, serum, or plasma obtained from the subject, as compared to a reference level of neurofilament light chain protein; and selecting the identified subject for treatment with a pharmaceutical composition comprising a therapeutically effective amount of a compound of formula (I), or a pharmaceutically acceptable salt, prodrug, or isomer thereof.

Also provided are methods of selecting a subject (e.g., any of the subjects described herein) for treatment that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject (e.g., any of the subjects described herein) for participation in a clinical trial that include: identifying a subject having an elevated level of neurofilament light chain protein in a sample comprising cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that comprises administration of a pharmaceutical composition comprising a therapeutically effective amount of a compound of formula (I), or a pharmaceutically acceptable salt, prodrug, or isomer thereof (e.g., CHS-131, e.g., using any of the doses or frequencies of administration described herein).

Also provided are methods of selecting a subject for participation in a clinical trial that include: identifying a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and selecting the identified subject for participation in a clinical trial that comprises administration of a pharmaceutical composition comprising a therapeutically effective amount of a compound of formula (I), or a pharmaceutically acceptable salt, prodrug, or isomer thereof (e.g., CHS-131, e.g., using any of the doses or frequencies of administration described herein).

### Methods of Predicting

Provided herein are methods of predicting the efficacy of a treatment in a subject that include: determining a level of neurofilament light chain protein level in a sample comprising cerebrospinal fluid, blood, serum, or plasma obtained from the subject; and determining that a treatment with a pharmaceutical composition comprising a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein, as compared to a subject not having an elevated level of neurofilament light chain protein in a sample comprising blood, serum, or plasma as compared to the reference level of neurofilament light chain protein.

In some aspects, the methods can include obtaining from the subject a second sample comprising cerebrospinal fluid, blood, serum, or plasma at a second time point and repeating the determining step.

### Compositions and Kits

Also provided herein are compositions (e.g., pharmaceutical compositions) that include at least one compound of formula (I) of any of the compounds described herein and instructions for performing any of the methods described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intracranial, intravenous, subcutaneous, or intramuscular). In some embodiments, the compositions (e.g., pharmaceutical compositions) can include a pharmaceutically acceptable salt (e.g., phosphate buffered saline). In some embodiments, the compositions (e.g., pharmaceutical compositions) can include a prodrug, or an isomer thereof. Single or multiple administrations of any of the pharmaceutical compositions described herein can be given to a subject depending on, for example: the dosage and frequency as required and tolerated by the patient. A dosage of the pharmaceutical composition should provide a sufficient quantity of the compound of formula (I) a pharmaceutically acceptable salt, prodrug, or an isomer thereof to effectively treat or ameliorate conditions, diseases or symptoms.

Also provided herein are kits containing one or more (e.g., at least 2, 3, 4, 5, 6, 7, 8, 9, 10, 12, 14, 16, 18, 20) of any of the pharmaceutical compositions described herein that include a therapeutically effective amount of a compound of formula (I), or a pharmaceutically acceptable salt, prodrug, or isomer thereof. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. The kits described herein are not so limited; other variations will be apparent to one of ordinary skill in the art.

### EXAMPLES

The invention is further described in the following examples, which do not limit the scope of the invention described in the claims.

### Example 1. Assay for Detecting Neurofilament Light Chain (NFL) in Serum, Plasma, or Blood Samples

Single-molecule array (Simoa) assay is a highly sensitive assay that allows accurate quantification of low neurofilament light chain concentrations (Disanto et al., Ann. Neurol. 81(6):857-870, 2017; Rohrer et al., Neurology 87(13):1329-1336, 2016; and Novakova et al., Neurology 89(22): 2230-2237, 2017).

Capture monoclonal antibody (mAB) 47: 3 is buffer exchanged and diluted to 0.3 mg/mL 4×10⁶ paramagnetic beads (Quanterix Corporation) are buffer exchanged and activated using 0.5 mg/mL 1-ethyl-3-(3-dimetylaminopropyl) carbodiimide (Quanterix), followed by a 30-minute incubation at room temperature (HulaMixer, Thermo Fischer Scientific). The diluted capture mAB 47:3 is conjugated with the washed and activated paramagnetic beads for a 2-hour incubation at room temperature on a mixer. After the incubation, the beads are washed three times using a magnetic separator and blocked. Next, the conjugated beads are suspended and stored at 4° C.

The Simoa assay is run on a Simoa HD-1 instrument (Quanterix) using a two-step Assay Neat 20 protocol. Briefly, 100 μL of calibrator/sample in Tris-buffered saline (TBS), 0.1% Tween 20, 1% milk powder, 400 μg/mL Heteroblock (Omega Biologicals), 25 μL conjugated beads in TBS, 0.1% Tween 20, 1% milk powder, 300 μg/mL Heteroblock, and 20 μL of mAB 2:1 (0.1 μg/mL in TBS, 0.1% Tween 20, 1% milk powder, 300 μg/mL Heteroblock) are incubated for 35 minutes 15 seconds (47 cadences with 1 cadence is 45 seconds). Next, the mixture is washed. Then, 100 μL of streptavidin-conjugated 3-galactosidase (150 pM; Quanterix) is added, followed by a 5 minutes 15 second (7-cadence) incubation and wash. The mixture will then be placed on a Simoa HD-1 instrument. Prior to reading, 25 μL Resorufin (3-D-galactopyranoside (Quanterix) is added. The calibrator curve is constructed using the standard from the NFL ELISA (NF-light, UmanDiagnostics). Samples and calibrators are measured in duplicates.

NFL levels are log-transformed to meet the normal assumption. The distribution of sNfl in healthy control is modeled by means of Generalized Additive Models for Location, Scale, and Shape (GAMLSS) using a Box-Cox t distribution according to Rigby and Stasinopoulous, Stat Med 23: 3053-3076, 2004, and cubic splines and percentile curves are obtained.

To quantify, intra- and interassay variability, bootstrapping is applied by drawing 100 random samples from the healthy controls.

Linear regression models are used to investigate the associations with log sNFL. Linear generalized estimating equation (GEE) models are similarly used to investigate associations with log sNFL.

### Example 2. Blood NFL Measurements by Ultrasensitive Simoa Assay

Serum Nfl levels were investigated in longitudinal blood samples. The aims of this study include:
to explore the predictive value of blood NFL concentrations in patients treated with CHS-131 (also known as INT-131),
to determine the kinetics of the response to CHS-131,
to analyze NFL as a marker for the effectiveness of CHS-131 in relapsing-remitting multiple sclerosis (RRMS) patients,
to assess the value of NFL concentrations in comparing the effectiveness of CHS-131 with the effectiveness of placebo,
to correlate blood NFL with radiological and clinical outcome parameters (such as magnetic resonance imaging (MRI) (Gd+ lesion counts, T2 lesion volume), annualized relapse rate (ARR), brain atrophy and correlation with expanded disability status scale (EDSS),
to examine the prognostic value of NFL concentrations by stratification of patients related to disease activity,
to determine the risk of relapse or risk of disease progression, and
to evaluate NFL concentrations as a retreatment predictive biomarker.

A commercially available ELISA (UmanDiagnostics NF-Light® assay) uses two highly specific, non-competing monoclonal antibodies (47:3 and 2:1) to quantify soluble NFL in cerebrospinal fluid (CSF) samples. Using this assay to analyze CSF samples, it was found that NFL is more sensitive than NFH to distinguish patients in different stages of MS versus controls (Kuhle et al., Mult Scler 19:1597-1603, 2013). Together with UmanDiagnostics a highly sensitive electrochemiluminescence (ECL)-based NfL assay was developed suitable for the quantification of NFL in serum at concentrations relevant to clinical settings (Gaiottino et al., PLoS One 2013; 8(9):e75091, 2013; and Disanto et al., J Neurol Neurosurg Psychiatry 87(2): 126-129, 2015). The HD-1 analyzer relies on single-molecule arrays (Simoa) and the simultaneous counting of singulated capture microbeads (Rissin et al., Nat Biotechnol 28:595-599, 2010). The instrument integrates established paramagnetic microbead-based reagent robotics with a novel imaging module that digitizes the immunoassay with an array consumable and optical system at a cost similar to that of conventional immunoassay platforms (Wilson et al., J Lab Autom 21(4): 533-547, 2015). The system can accommodate user-developed custom reagents and assay protocols, as well as a menu of pre-validated assay kits. Across a range of immunoassays covering several disease areas, the fully automated Simoa assays exhibited ≥4 logs of measurement range, single digit CVs, and sensitivities in the femtogram per milliliter range (Wilson et al., J Lab Autom 21(4): 533-547, 2015). Thus, the Simoa HD-1 Analyzer is approximately 3 logs more sensitive than conventional fluorescence, chemiluminescence, and ECL immunoassay instrumentation.

Patients with presumed sampling at baseline and patients with presumed sampling at 12 and 24 weeks from the placebo group, or the 1 mg CHS-131 or 3 mg CHS-131 groups were tested. All patients receiving CHS-131 were orally adminisntered 1 mg or 3 mg CHS-131 daily. All control patients were orally administered a placebo tablet once a day. All patients enrolled in the study were treatment-naïve RRMS for <3 years.

The human plasma samples used for the analysis were coded and are not individually identifiable because neither the investigator nor any other individuals associated with the investigation or the sponsor can link the specimen to the subject from whom the specimen was collected, either directly or indirectly through coding systems.

The concentration of NFL (ng/mL) in subject administered placebo, 1 mg daily CHS-131, and 3 mg daily CHS-131 are reported in Tables 1, 2, and 3, respectively.

TABLE 1

| | Wk0 | Wk12 | Wk24 | Wk48 |
|---|---|---|---|---|
| n | 4 | 74 | 72 | 65 |
| Mean | 19.18 | 27.88 | 23.49 | 18.62 |
| SD | 5.667 | 35.098 | 24.256 | 12.570 |
| Median | 20.6 | 17.15 | 15.9 | 15.9 |
| Q1 | 15.35 | 10.8 | 9.1 | 10.8 |
| Q3 | 23 | 27.2 | 30.1 | 20.9 |
| Min | 11.2 | 2.6 | 2.7 | 4.9 |
| Max | 24.3 | 224.5 | 169 | 62.4 |

TABLE 2

| | Wk0 | Wk12 | Wk24 | Wk48 |
|---|---|---|---|---|
| n | 5 | 75 | 74 | 69 |
| Mean | 20.76 | 27.56 | 22.29 | 27.62 |
| SD | 5.738 | 27.459 | 15.750 | 43.699 |
| Median | 19.4 | 17.9 | 17 | 17.5 |
| Q1 | 19.3 | 11.7 | 11.8 | 10.1 |
| Q3 | 22.8 | 32.2 | 31.5 | 24.2 |
| Min | 13.3 | 2.1 | 3.2 | 2.6 |
| Max | 29 | 159.5 | 65.5 | 340.3 |

TABLE 3

| | Wk0 | Wk12 | Wk24 | Wk48 |
|---|---|---|---|---|
| n | 7 | 72 | 72 | 67 |
| Mean | 29.86 | 23.17 | 20.38 | 19.33 |
| SD | 23.117 | 17.895 | 14.375 | 18.235 |
| Median | 19.6 | 16.45 | 15.9 | 14.3 |
| Q1 | 11.8 | 12.25 | 10.4 | 9 |
| Q3 | 48.1 | 28.15 | 26.55 | 20.4 |
| Min | 11.7 | 4.9 | 5.8 | 4.9 |
| Max | 73.7 | 117.1 | 74.9 | 118.7 |

The correlation between PK concentration and NFL concentration was evaluated at time points for the placebo group, the 1 mg daily CHS-131 group, and the 3 mg daily CHS-131 group. Pearson, Spearmen, and Kendall correlations were evaluated. The correlations are reported for week 12, week 24, and week 48 in Tables 4, 5, and 6, respectively.

TABLE 4

| Week 12 | | | | | |
|---|---|---|---|---|---|
| Placebo | | INT131 1 mg QD | | INT131 3 mg QD | |
| Corre- | P- | Corre- | P- | Corre- | P- |
| lation | value | lation | value | lation | value |

| | | | | |
|---|---|---|---|---|
| Pearson | 0.1288 | 0.2775 | −0.2166 | 0.0739 |
| Spearman | 0.1665 | 0.1592 | −0.1264 | 0.3008 |
| Kendall | 0.1117 | 0.1629 | −0.0870 | 0.2929 |

TABLE 5

| | Week 24 | | | | | |
|---|---|---|---|---|---|---|
| | Placebo | | INT131 1 mg QD | | INT131 3 mg QD | |
| | Corre- | P- | Corre- | P- | Corre- | P- |
| | lation | value | lation | value | lation | value |
| Pearson | 0.0390 | 0.7523 | 0.1360 | 0.2616 | −0.0662 | 0.5860 |
| Spearman | 0.1338 | 0.2766 | 0.0919 | 0.4491 | −0.0515 | 0.6721 |
| Kendall | 0.1101 | 0.2733 | 0.0544 | 0.5065 | −0.0311 | 0.7037 |

TABLE 6

| | Week 48 | | | | | |
|---|---|---|---|---|---|---|
| | Placebo | | INT131 1 mg QD | | INT131 3 mg QD | |
| | Corre- | P- | Corre- | P- | Corre- | P- |
| | lation | value | lation | value | lation | value |
| Pearson | −0.0853 | 0.5686 | 0.0982 | 0.4843 | −0.1371 | 0.3526 |
| Spearman | −0.0025 | 0.9869 | 0.0745 | 0.5962 | −0.2446 | 0.0938 |
| Kendall | 0.0121 | 0.9051 | 0.0483 | 0.6123 | −0.1658 | 0.0981 |

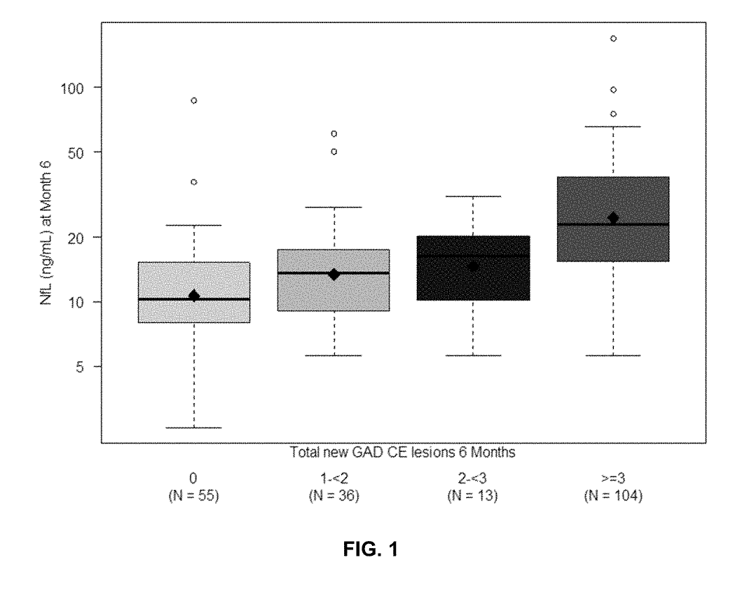

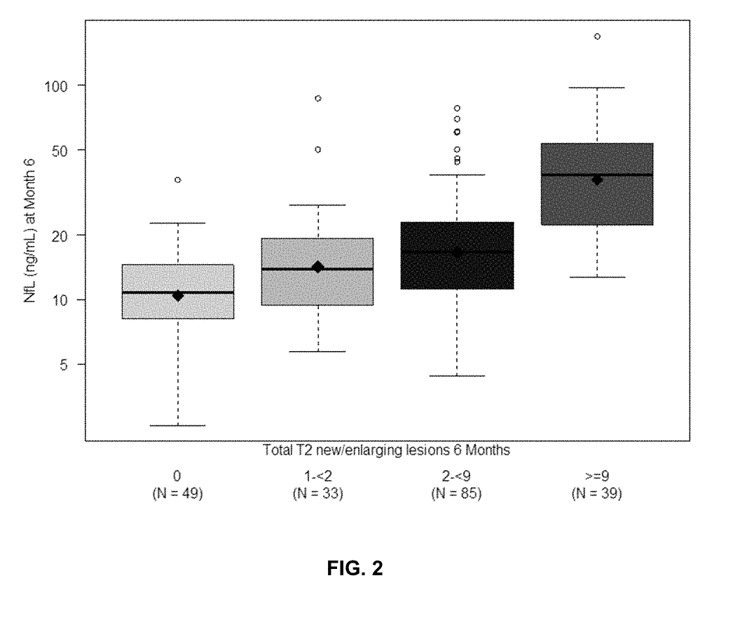

The correlation between NFL concentration and CE and T2 lesions was evaluated after six months of treatment with CHS-131 (all groups combined). FIG. 1 shows subjects administered CHS-131 that had lower average levels of NFL had fewer new CE lesions. FIG. 2 shows subjects administered CHS-131 that had lower average levels of NFL had fewer new or enlarging T2 lesions. Since reducing CE lesions and T2 lesions is considered effective treatment of multiple sclerosis, these results show that reduced NFL levels in subjects administered CHS-131 is indicative of effective treatment of multiple sclerosis.

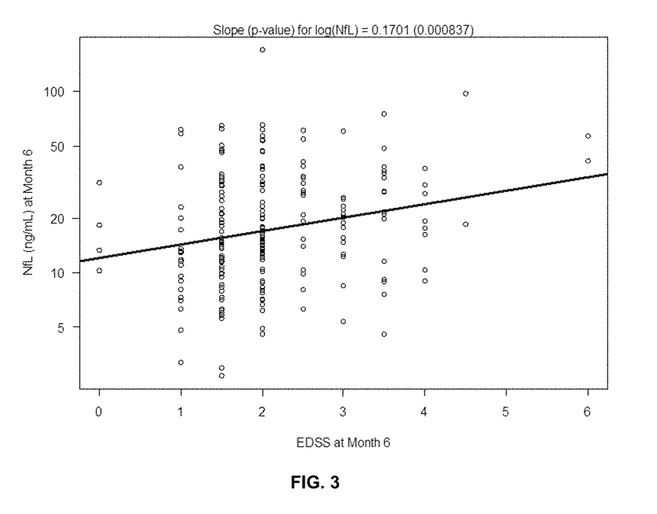

The correlation between NFL concentration and EDSS was evaluated after six months of treatment with CHS-131 (all groups combined). FIG. 3 shows a trend that subjects administered CHS-131 that had lower levels of NFL lower EDSS. Increased EDSS indicates increased disability, therefore lowering EDSS is considered effective treatment of multiple sclerosis. Since, lowering EDSS is considered effective treatment of multiple sclerosis, these results show that reduced NFL levels in subjects administered CHS-131 is indicative of effective treatment of multiple sclerosis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject, the method comprising:
selecting a subject having an elevated level of neurofilament light chain protein in a sample comprising cerebrospinal fluid, blood, serum, or plasma obtained from the subject, as compared to a reference level of neurofilament light chain protein; and
administering a pharmaceutical composition comprising a therapeutically effective amount of a compound of formula (I), 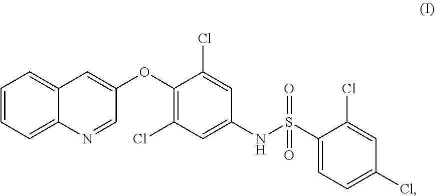 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, to the selected subject.

2. A method of selecting a treatment for a subject, the method comprising:
identifying a subject having an elevated level of neurofilament light chain protein in a sample comprising cerebrospinal fluid, blood, serum, or plasma obtained from the subject, as compared to a reference level of neurofilament light chain protein; and
selecting a pharmaceutical composition comprising a therapeutically effective amount of a compound of formula (I), 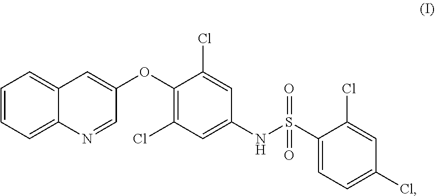 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, for the identified subject.

3. A method of selecting a subject for treatment, the method comprising:
identifying a subject having an elevated level of neurofilament light chain protein in a sample comprising cerebrospinal fluid, blood, serum, or plasma obtained from the subject, as compared to a reference level of neurofilament light chain protein; and
selecting the identified subject for treatment with a pharmaceutical composition comprising a therapeutically effective amount of a compound of formula (I), 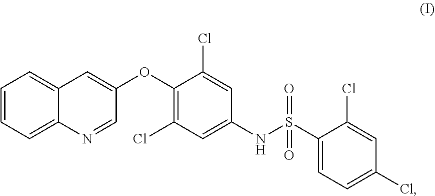 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

4. A method of selecting a subject for participation in a clinical trial, the method comprising:
identifying a subject having an elevated level of neurofilament light chain protein in a sample comprising cerebrospinal fluid, blood, serum, or plasma obtained from the subject, as compared to a reference level of neurofilament light chain protein; and
selecting the identified subject for participation in a clinical trial that comprises administration of a pharmaceutical composition comprising a therapeutically effective amount of a compound of formula (I), 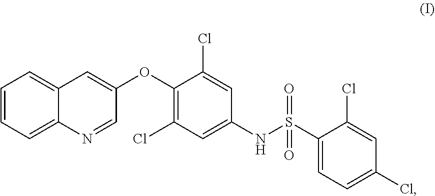 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

5. A method of predicting the efficacy of a treatment in a subject, the method comprising:
determining a level of neurofilament light chain protein level in a sample comprising cerebrospinal fluid, blood, serum, or plasma obtained from the subject; and
determining that a treatment with a pharmaceutical composition comprising a therapeutically effective amount of a compound of formula (I), 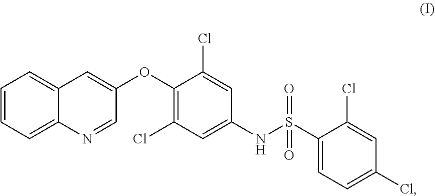 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, is more likely to be effective in a subject having an elevated level of neurofilament light chain protein in the sample as compared to a reference level of neurofilament light chain protein, as compared to a subject not having an elevated level of neurofilament light chain protein in a sample comprising blood, serum, or plasma as compared to the reference level of neurofilament light chain protein.

6. The method of any one of claims 1-5, wherein the subject has not been diagnosed with a neurological disorder or neural tissue damage.

7. The method of any one of claims 1-6, wherein the subject does not present with a symptom of a neurological disorder or neural tissue damage.

8. The method of any one of claims 1-5, wherein the subject has been diagnosed as having a neurological disorder or neural tissue damage.

9. The method of any one of claims 1-8, wherein the method further comprises performing an assay to determine the level of neurofilament light chain protein in the sample obtained from the subject.

10. The method of claim 9, wherein the assay is a single-molecule array assay.

11. The method of claim 2, wherein the method further comprises administering the selected treatment to the identified subject.

12. The method of any one of claims 1-11, wherein the subject has been previously administered a different pharmaceutical composition and the different pharmaceutical composition was determined not to be therapeutically effective.

13. A method of determining the efficacy of a treatment in a subject, the method comprising:
determining a first level of neurofilament light chain protein level in a sample comprising cerebrospinal fluid, blood, serum, or plasma obtained from the subject at a first time point;
determining a second level of neurofilament light chain protein level in a sample comprising cerebrospinal fluid, blood, serum, or plasma obtained from the subject at a second time point, wherein the subject received at least one dose of a pharmaceutical composition comprising a therapeutically effective amount of a compound of formula (I), 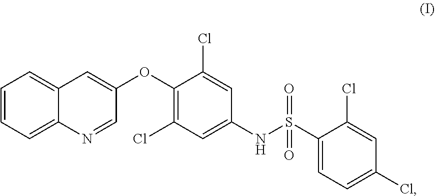 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, between the first and the second time points; and
identifying the pharmaceutical composition as being effective in a subject having a reduced second level of neurofilament light chain protein as compared to the first level of neurofilament light chain protein.

14. The method of claim 13, wherein the subject is a participant in a clinical trial.

15. The method of claim 13 or 14, wherein the method further comprises administering one or more additional doses of the pharmaceutical composition identified as being effective to the subject.

16. A method of treating a subject, the method comprising:
selecting a subject having an elevated second level of neurofilament light chain protein in a sample comprising cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and
administering a pharmaceutical composition comprising a therapeutically effective amount of a compound of formula (I), 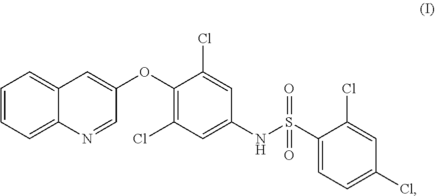 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, to the selected subject.

17. A method of selecting a treatment for a subject, the method comprising:
identifying a subject having an elevated second level of neurofilament light chain protein in a sample comprising cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and
selecting a pharmaceutical composition comprising a therapeutically effective amount of a compound of formula (I), 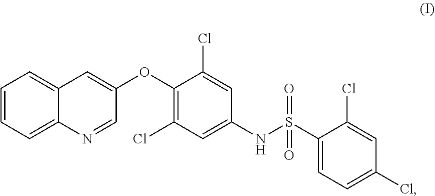 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof, for the identified subject.

18. A method of selecting a subject for treatment, the method comprising:
identifying a subject having an elevated second level of neurofilament light chain protein in a sample comprising cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and
selecting the identified subject for treatment with a pharmaceutical composition comprising a therapeutically effective amount of a compound of formula (I), 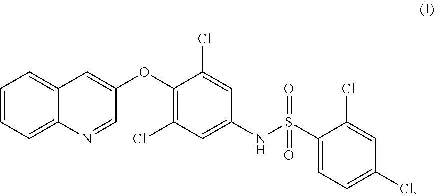 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

19. A method of selecting a subject for participation in a clinical trial, the method comprising:
identifying a subject having an elevated second level of neurofilament light chain protein in a sample comprising cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and
selecting the identified subject for participation in a clinical trial that comprises administration of a pharmaceutical composition comprising a therapeutically effective amount of a compound of formula (I), 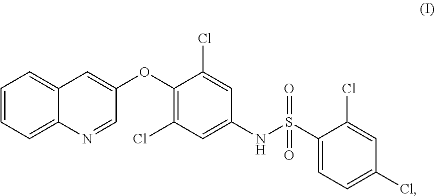 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

20. The method of any one of claims 13-19, wherein the subject has not been diagnosed with a neurological disorder or neural tissue damage.

21. The method of any one of claims 13-20, wherein the subject does not present with a symptom of a neurological disorder or neural tissue damage.

22. The method of any one of claims 13-19, wherein the subject has been diagnosed as having a neurological disorder or neural tissue damage.

23. The method of any one of claims 13-22, wherein the method further comprises performing an assay to determine the first level and second levels of neurofilament light chain protein in the sample obtained from the subject at the first time point and the second time point, respectively.

24. The method of claim 23, wherein the assay is a single-molecule array assay.

25. The method of claim 17, wherein the method further comprises administering the selected treatment to the identified subject.

26. The method of any one of claims 13-25, wherein the subject has been previously administered a different pharmaceutical composition and the different pharmaceutical composition was determined not to be therapeutically effective.


SEQUENCE LISTING (4 sequences)

SEQ ID NO: 1 - DNA, 1632 bp, Homo sapiens
atgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattga

SEQ ID NO: 2 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 3 - DNA, 1632 bp, Mus musculus
atgagttcgttcggctacgatccgtacttttcgacctcctacaagcggcgctatgtggagacgccccgggtgcacatctccagcgtgcgcagcggctacagcacggcgcgctccgcgtactccagctactccgcgccggtctcctcctcgctgtccgtgcgccgcagctactcgtccagctctggctctttgatgcccagcctggagaatctcgatctgagccaggtagccgccatcagcaacgacctcaagtctatccgcacacaagagaaggcacagctgcaggacctcaacgatcgcttcgccagcttcatcgagcgcgtgcacgagctggagcagcagaacaaggtcctggaagccgagctgttggtgctgcgccagaaacactctgagccttcccgcttccgcgccctgtacgagcaggagatccgcgatctgcggctggcagcggaagacgccactaacgagaagcaggcgctgcagggcgagcgcgaggggctggaggagactctgcgcaacctgcaggctcgctatgaggaagaagtgctgagccgcgaggacgccgagggccggctgatggaagcgcgcaaaggtgcggatgaggccgcgctcgcccgcgccgagctggagaagcgcatcgacagcctgatggacgagatagctttcctgaagaaggtgcacgaggaagagatcgccgagctgcaggctcagatccagtatgctcagatctccgtggagatggacgtgtcctccaagcccgacctctccgccgctctcaaggacatccgcgctcagtacgagaagctggccgccaagaacatgcagaacgccgaagagtggttcaagagccgcttcaccgtgctaaccgagagcgccgccaagaacaccgacgctgtgcgcgctgccaaggacgaggtgtcggaaagccgccgcctgctcaaggctaagaccctggagatcgaagcctgccggggtatgaacgaagctctggagaagcagctgcaggagctagaggacaagcagaatgcagacattagcgccatgcaggacacaatcaacaaactggagaatgagctgagaagcacgaagagcgagatggccaggtacctgaaggagtaccaggacctcctcaatgtcaagatggccttggacatcgagattgcagcttacagaaaactcttggaaggcgaagagaccaggctcagtttcaccagcgtgggtagcataaccagcggctactctcagagctcgcaggtcttcggccgttctgcttacagtggcttgcagagcagctcctacttgatgtctgctcgctctttcccagcctactataccagccacgtccaggaagagcagacagaggtcgaggagaccattgaggctacgaaagctgaggaggccaaggatgagcccccctctgaaggagaagcagaagaggaggagaaggagaaagaggagggagaggaagaggaaggcgctgaggaggaagaagctgccaaggatgagtctgaagacacaaaagaagaagaagaaggtggtgagggtgaggaggaagacaccaaagaatctgaagaggaagagaagaaagaggagagtgctggagaggagcaggtggctaagaagaaagattga

SEQ ID NO: 4 - protein, 543 aa, Mus musculus
MSSFGYDPYFSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEIAFLKKVHEEEIAELQAQIQYAQISVEMDVSSKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRSTKSEMARYLKEYQDLLNVKMALDIEIAAYRKLLEGEETRLSFTSVGSITSGYSQSSQVFGRSAYSGLQSSSYLMSARSFPAYYTSHVQEEQTEVEETIEATKAEEAKDEPPSEGEAEEEEKEKEEGEEEEGAEEEEAAKDESEDTKEEEEGGEGEEEDTKESEEEEKKEESAGEEQVAKKKD